(19) United States
(12) Patent Application Publication
(10) Pub. No.: US 2019/0255356 A1
(43) Pub. Date: Aug. 22, 2019

(54) Method and System for Treatment of a Patient's Tumor

(71) Applicant: Landeskrankenanstalten-Betriebsgesellschaft - KABEG, Klagenfurt am Worthersee (AT)

(72) Inventor: Slavisa Tubin, Klagenfurt am Worthersee (AT)

(21) Appl. No.: 15/902,011

(22) Filed: Feb. 22, 2018

(51) Int. Cl. A61N 5/10; A61B 6/03; A61B 6/00; G16H 20/40

(52) U.S. Cl. CPC A61N 5/1039; A61B 6/037; G16H 20/40; A61B 6/5217; A61B 6/5235; A61B 6/032

(57) ABSTRACT

The disclosed subject-matter relates to a method for treatment of a patient by irradiating a tumor by means of an irradiation device, the method comprising the steps of scanning at least a part of a body of the patient to generate a scan, said part comprising the tumor; localizing the tumor in the scan; determining a hypoxic and a normoxic region of the tumor in the scan; targeting only the hypoxic region for irradiation; and irradiating the targeted hypoxic region; wherein the normoxic region is not targeted for irradiation during the treatment. The disclosed subject-matter further relates to a system configured to carry out the method.

### TECHNICAL FIELD

The disclosed subject-matter relates to a method for treatment of an oncological patient by irradiating a tumor by means of an irradiation device. In a further aspect, the disclosed subject-matter relates to a system for executing said method.

### BACKGROUND

Traditional radiobiology is based on the theory that the effects of radiotherapy present themselves only within an irradiated tissue of a patient, through direct and indirect DNA damage (Cook A. M., Berry R. J., Direct and indirect effects of irradiation: their relation to growth, Nature, 1966, 210:324-325).

Currently, after sporadic reports that stated that irradiation effects occasionally occur outside the irradiated tissue, the local effects were denominated as targeted effects, and the distant ones as non-targeted effects (Nagasawa H., Little J. B., Induction of sister chromatid exchanges by extremely low irradiation doses of alpha-particles, Cancer Res, 1992, 52:6394-6396).

Two types of non-targeted effects were described, depending on the site of their occurrence and the relationship between the irradiated and non-irradiated tumor:

The Radiation-Induced Abscopal Effect (RIAE) is a systemic effect of local irradiation, which means it extends to distant non-irradiated tissues outside the treated tissue. Basically, this means that tumor regression is observed at distant untreated sites.

The Radiation-Induced Bystander Effect (RIBE) is a radiobiological effect-transmission that happens when irradiation of only a part of the tumor induces regression of the whole tumor.

However, both phenomena have only been sporadically clinically observed, as they were occasional and unintentional and it was not possible to induce RIAE/RIBE on a consistent basis.

### SUMMARY

It is an object of the disclosed subject-matter to provide an effective method and system for treatment of a patient, for example metastatic patients with large (bulky) tumors, by irradiating a tumor.

To this end, in a first aspect the disclosed subject-matter provides for a method for treatment of a patient by irradiating a tumor by means of an irradiation device, the method comprising the following steps: scanning at least a part of a body of the patient to generate a scan, said part comprising the tumor; localizing the tumor in the scan; determining a hypoxic and a normoxic region of the tumor in the scan; targeting only the hypoxic region for irradiation; and irradiating the targeted hypoxic region; wherein the normoxic region is not targeted for irradiation during the treatment.

For the control and eradication of a tumor, the state of art deems it necessary to cover the entire tumor with the prescribed irradiation dose, including its possible microscopic infiltration ((Sub-)Clinical Target Volume CTV) outside the detectable tumor, and that the prescribed irradiation dose should be as homogeneous as possible (at least 95%). Furthermore, the larger the tumor the higher the irradiation dose should be prescribed in order to increase the probability of killing all tumor cells. In a case of large (bulky) tumors, due to their very high volume that has to be irradiated, with current methods it is not possible to deliver the necessary high irradiation dose as such a treatment would induce more damage to the healthy tissue than to the tumor. That is why current conventional radiotherapy technique finds its application only as a palliative treatment with an aim to control symptoms induced by bulky tumors.

In contrast thereto, targeting only the hypoxic region, as in the present method, has the effect that the overwhelming amount of the irradiation dose of the irradiation treatment is thus applied to the hypoxic region of the tumor with only little loss to the normoxic region. When applying the present method, it has been surprisingly experienced that RIAE/RIBE occurs fairly consistently when the surrounding peripheral normoxic tumor region and healthy tissues are not primarily subjected to irradiation.

Results have shown that irradiation with high-dose radiotherapy of only a part of a tumor, namely the hypoxic region, induces an intense RIAE/RIBE. Thereby, targeting exclusively the hypoxic region during irradiation leads to partial and/or complete long lasting regression of bulky tumor but also of un-irradiated metastatic disease, which yields a very effective tumor treatment.

Furthermore, as the irradiation dose outside the partially irradiated tumor is very low by using the technique described herein, it allows for a very safe re-irradiation in case of relapse; the down-sizing of the voluminous tumors by employing RIBE has the potential to convert non-resectable into resectable tumors, and a palliative intent into a curative intent.

The way of irradiating the hypoxic region of the tumor can be chosen depending on the available setup for irradiation, for example by means of three-dimensional conformal radiotherapy (3D-CRT) with multi-leaf-collimator, intensity modulated radiation therapy (IMRT), volumetric modulated arc therapy (VMAT), or with a photon, electron, or heavy charged particle beam. These irradiation methods can be classified in two main categories, which will be outlined in the following.

In the first category of irradiation devices, the irradiation device emits irradiation beams in different irradiation directions that intersect at the targeted hypoxic region, wherein said step of targeting is performed by setting an aperture, e.g., a multi-leaf collimator, of the irradiation device to match a contour of the hypoxic region of the tumor as seen in the respective irradiation direction, wherein said step of irradiating is performed by emitting irradiation beams in the respective irradiation direction through the aperture onto the targeted hypoxic region, the aperture blocking irradiation beams from irradiating the normoxic region outside said contour, and wherein the steps of targeting and irradiating are performed for each irradiation direction of the treatment.

This embodiment has the advantage that irradiation devices can be utilized that emit irradiation beams having a diameter that is larger than a typical hypoxic region of a tumor. Such irradiation devices usually have one irradiation source emitting a single irradiation beam, and the source can be rotated about the patient's body, usually having the tumor or the hypoxic region of the tumor, respectively, as a center for rotation.

In the second category of irradiation devices, the irradiation device emits irradiation beams in different irradiation directions that intersect at a single movable irradiation spot, wherein said step of targeting is performed by moving the irradiation spot onto a target point, wherein the step of irradiating is performed by emitting said irradiation beams in all of said different irradiation directions onto said target point, and wherein said steps of targeting and irradiating are performed for each of a plurality of different target points defined in the hypoxic region, wherein no target points are defined in the normoxic region during the treatment.

This embodiment has the advantage that irradiation devices can be utilized that emit one or more irradiation beams having a diameter smaller, usually at least a tenth smaller, than a typical hypoxic region of a tumor. The irradiation device can for this purpose have multiple irradiation sources emitting the irradiation beams in different directions, or again only a single irradiation source that rotates about the patient for generating irradiation beams in different directions.

In one embodiment, a positron emission tomography is used to generate the scan. Hence, the tumor and the hypoxic and normoxic regions can readily be identified. To this end, a standardized uptake value of the positron emission tomography scan is calculated and the hypoxic region is determined as a region in which the standardized uptake value, e.g., in a case of using 18F-FDG as a tracer, is equal to or smaller than 3. The region with the standard uptake value of 3 or less usually corresponds to an unvascularized or hypovascularized part of the tumor, which correlates to the hypoxic region.

Depending on the embodiment and tumor type, a tracer 18F-FMISO or 18F-FDG is used for positron emission tomography. Tracer 18F-FMISO is a hypoxia-specific tracer, meaning that a hypoxic region can be detected with a high degree of certainty, and is thus well suited for the method. Furthermore, tracer 18F-FDG having an acceptable predictive value for tumor hypoxia can be used especially among some aggressive tumor types like in those patients with gastric carcinoma, tongue cancer, non-small cell lung cancer (NSCLC), or oral squamous cell carcinoma.

In another embodiment, a computed tomography, which is optionally contrast enhanced, is used to generate the scan by combining a result of the positron emission tomography with a result of the computed tomography.

In the above-mentioned method, an irradiation dose of at least 8 Gy can be used for irradiation during the treatment, i.e., in a single fraction. The treatment can also be repeated a predefined number of times, e.g., 3 times, in consecutive days or after a lapse of a predefined period of time, for example after 7 days.

In a second aspect, the disclosed subject-matter provides for a system for carrying out the above-mentioned method. The system can be utilized with the same embodiments and brings about the same effects and advantages as described above for the method.

### BRIEF DESCRIPTION OF THE DRAWINGS/FIGURES

The disclosed subject-matter shall now be explained in more detail below on the basis of exemplary embodiments thereof with reference to the accompanying drawings, in which:

FIG. 1 shows a system for irradiation treatment of a patient, in a block diagram,

FIGS. 2a to 2c show, in a first embodiment of the system of FIG. 1, a first (FIG. 2a) and a second step (FIG. 2b) of the method of treatment in a schematic cross-section, and an aperture of an irradiation device of the system (FIG. 2c) in a plan view,

FIGS. 3a, 3b show, in a second embodiment of the system of FIG. 1, a first (FIG. 3a) and a second step (FIG. 3b) of the method of treatment in a schematic cross-section, and

FIGS. 4a-4e show different cross-sections of a patient's thoracic cavity with a voluminous squamous cell carcinoma in the right lung (FIG. 4a), distant mediastinal lymphadenopathies/metastases (FIG. 4b), an induction of a bystander effect by targeting the hypoxic region (FIG. 4c), great reduction of whole partially treated tumor due to a bystander effect-induction after three weeks (FIG. 4d), and disappearance of untreated distant mediastinal lymphadenopathies due to an abscopal effect (FIG. 4e).

### DETAILED DESCRIPTION

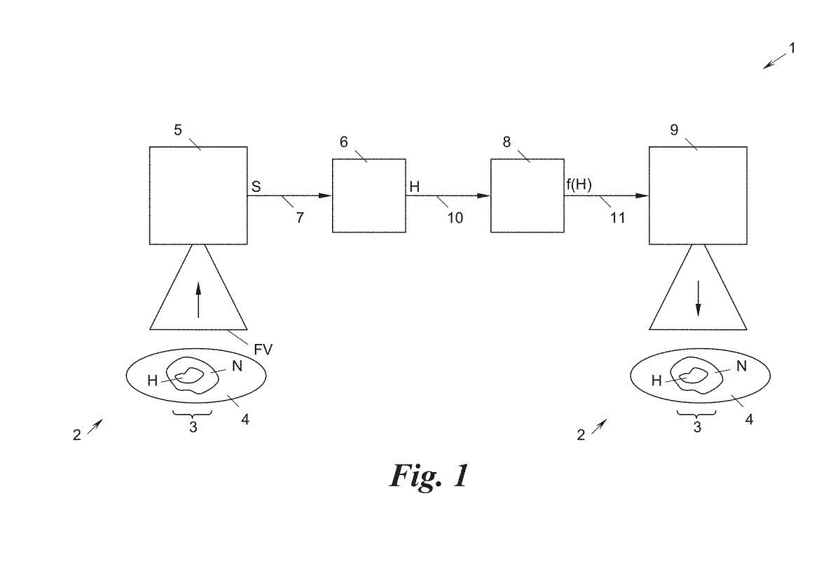

FIG. 1 shows a system 1 for treatment of a patient 2 by irradiating a tumor 3, wherein only a cross-section of the patient's body 4 is depicted in FIG. 1. As known in the state of the art, tumors 3 of the type discussed herein have a hypoxic region H and a normoxic region N. The hypoxic region H is a region that usually comprises a hypovascularized and hypometabolic tumor segment, while the normoxic region N has a hypermetabolic tumor segment. In such tumors 3, the hypoxic region H is commonly surrounded by the normoxic region N.

For localizing the tumor 3 within the patient's body 4, the system comprises a scanner 5, which scans at least a part of the body 4 of the patient 2 to generate a scan S. The generated scan S is a three-dimensional (3D) scan such that a 3D model of the part of the body 4 and of the tumor 3 can be generated. Depending on the embodiment, the 3D scan S can also be composed of a multitude of two-dimensional (2D) scans.

The scan S can be generated by any method known in the art, for example by positron emission tomography or computed tomography. Said methods can also be combined to obtain a higher-quality scan S, for example by combining a result of the positron emission tomography with a result of the computed tomography. For positron emission tomography, a tracer can be used, for example tracer 18F-FMISO or 18F-FDG, which are especially suited for detecting the hypoxic region H as outlined below.

The scan S can be a scan of the whole body 4 of the patient but also only a partial scan S of the body 4, i.e., the part of the body 4 comprising the tumor 3. For this purpose, the scanner 5 can have an adjustable field of view FV which can either be set mechanically or computationally.

The scanner 5 is connected to a processor 6 for receiving the scan S by means of a connection 7, for example a wired or wireless (WiFi, Bluetooth, RFID, et cet.) direct connection or an indirect connection such as a USB storage medium for the scan S that can be connected to interfaces on both the scanner 5 and processor 6.

After receiving the scan S, the processor 6 localizes the tumor 3 therein and determines the hypoxic region H and the normoxic region N of the tumor 3 in the scan S, again as 3D regions. This can be done by means of any method known in the art, for example by measuring a standard uptake value (SUV) of the scan S created by positron emission tomography with the abovementioned tracers. A SUV of, e.g., 3 or less may indicate the hypoxic region H while a standard uptake value larger than 3 within the tumor would consequently indicate the normoxic region N.

The system 1 further comprises a controller 8 and an irradiation device 9. The controller 8 is connected to the processor 6 by means of a connection 10 that can be of the types as the abovementioned connection 7. The controller 8 and the processor 6 can be separate from each other or alternatively be integrated in one and the same physical entity or even be implemented as different tasks/applications in one and the same processing device. In another embodiment, the controller 8 is a part of the irradiation device 9.

The controller 8 controls the irradiation device 9 via a connection 11 that can also be of any type mentioned above with respect to the connection 7. The controller 8 has the purpose of interacting with actuators or drive components of the irradiation device 9 to adjust the irradiation device 9 to target only the hypoxic region H determined by the processor 6, whereof the controller 8 received the information on the hypoxic region H for controlling the irradiation device 9. This is symbolically shown as control function f(H) of the controller 8.

Thereafter, the irradiation device 9 irradiates the targeted hypoxic region H.

The system 1 only targets the hypoxic region H for irradiation. During the whole treatment, which can take several minutes (usually not more than 20 minutes), depending, inter alia, on the irradiation device 9, the normoxic region N is not targeted for irradiation.

During the treatment, the irradiation device 9 delivers an irradiation dose of, e.g., at least 8 Gy for irradiation of the hypoxic region H, i.e., in a single fraction.

The irradiation device 9 can be of any type known in the state of the art, for example it can be operated by means of three-dimensional conformal radiotherapy (3D-CRT) with multi-leaf collimator, intensity modulated radiation therapy (IMRT), volumetric modulated arc therapy (VMAT), or a photon, electron, or heavy charged particle beam. Different ways of irradiating are disclosed in U.S. Pat. No. 8,395,131 B2, for example, which is hereby incorporated by reference.

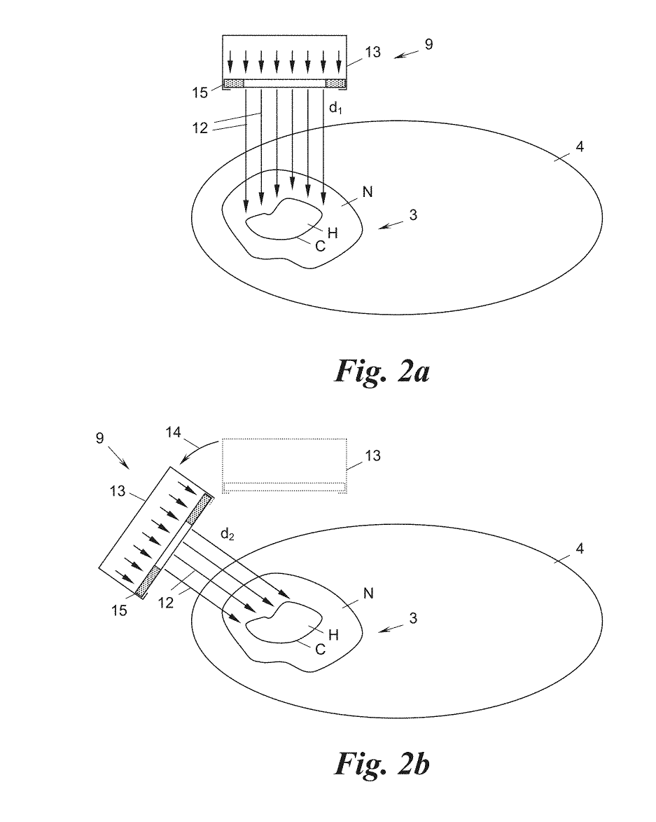

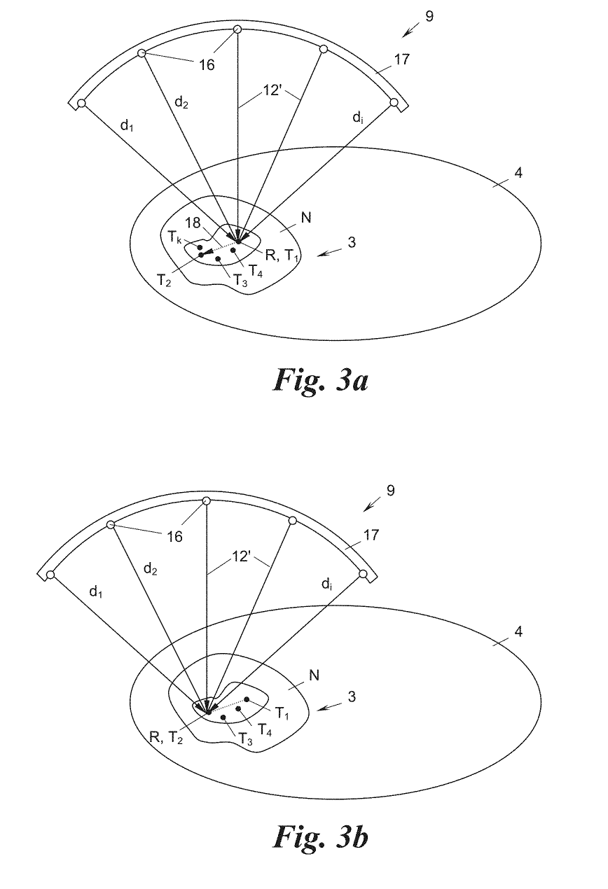

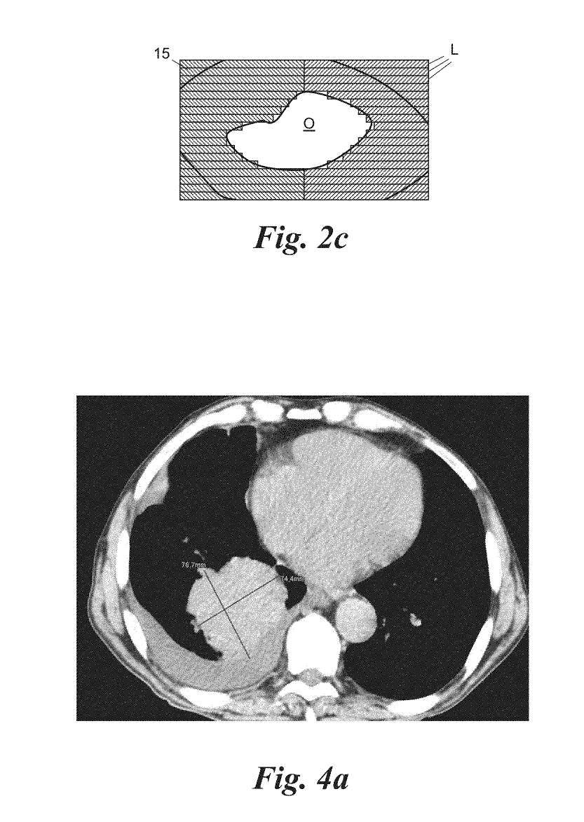

FIGS. 2a and 2b show a type of irradiation device 9 that emits irradiation beams 12 in different irradiation directions d₁, d₂, . . . , generally dᵢ, that intersect at the hypoxic region H. Specifically, in FIG. 2a the irradiation device 9 emits irradiation beams 12 in a first irradiation direction d₁ onto the hypoxic region H, while in FIG. 2b the irradiation device 9 emits irradiation beams 12 in a second irradiation direction d₂ onto the hypoxic region H after the irradiation device 9, or at least its head 13, was rotated about the body 4 of the patient 2 (arrow 14).

To ensure that only the hypoxic region H and not the normoxic region N is targeted throughout the treatment, the irradiation device 9 comprises an aperture 15, which can for example be embodied as a collimator. The targeting is performed by setting the aperture 15 of the irradiation device 9 to match a contour C of the hypoxic region H of the tumor 3 as seen in the irradiation direction dᵢ. To this end, the opening of the aperture 15 is variable.

FIG. 2c shows an example of such an aperture 15. The aperture 15 of this example comprises longitudinal cover strips L that can be inserted into and retracted from the aperture 15 from both sides to variably create an opening O that matches the contour C of the hypoxic region H as seen in the irradiation direction dᵢ. An alternative could be an aperture 15 that is embodied as holes that are arranged in an array, whereupon holes can be selectively covered such that the opening O matches the shape of the contour C.

After the aperture 15 has been set to match the contour C of the hypoxic region H, the irradiation device 9 performs the step of irradiating by emitting irradiation beams 12 in the first irradiation direction d₁ through the aperture 15 onto the targeted hypoxic region H. The aperture 15 thus blocks irradiation beams 12 from irradiating the normoxic region N outside said contour C.

After irradiating in the first irradiation direction d₁, the irradiation device 9 (or its head 13) rotates so that it can irradiate the hypoxic region H from the second irradiation direction d₂ which is different from the first irradiation direction d₁.

When necessary for the treatment of the patient 2, the irradiation device 9 continues to irradiate the hypoxic region H in further irradiation directions d₃, d₄, . . . , dᵢ, . . . until it will deliver a full planned irradiation dose to the hypoxic region. The controller 8 and the irradiation device 9 are configured to perform the targeting and irradiating for each respective irradiation direction dᵢ of the treatment as described above.

FIG. 3a shows an exemplary embodiment of a different type of irradiation device 9. In the shown embodiment, the irradiation device 9 consists of a multitude of irradiation sources 16 that are fixated on a mounting 17. The individual irradiation sources 16 each emit an irradiation beam 12′ in a respective different irradiation direction d₁, d₂, . . . , dᵢ, . . . , such that the irradiation beams 12′ intersect at a single movable irradiation spot R. The irradiation beams 12′ each have a cross section that is smaller than a typical diameter of the hypoxic region H to be treated, e.g., 10 times smaller. Hence, also the irradiation spot R is smaller than the typical diameter of the hypoxic region H.

Alternatively to the multiple irradiation sources 16 on the mounting 17, again a single irradiation source 16 could be used that rotates about the irradiation spot R such that each of the emitted irradiation beams 12′ intersect at an irradiation spot R.

For such an irradiation device 9 using irradiation beams 12′ of a small cross section, the controller 8 defines, before targeting, a plurality of different target points T₁, T₂, . . . , Tₖ, . . . , T_{K}, within the hypoxic region H. No target points Tₖ are defined in the normoxic region N during the treatment to exclusively target the hypoxic region H. Typically, the target points Tₖ are defined such that they have a predefined density in the hypoxic region H, e.g., 1 target point Tₖ per cm³.

For targeting the hypoxic region H, the controller 8 “moves” the irradiation spot R onto the first target point T₁, i.e., selects the first target point T₁ for irradiation, as shown in FIG. 3a, whereupon the irradiation device 9 emits said irradiation beams 12 in all of said different irradiation directions dᵢ onto said first target point T₁.

The moving of the irradiation spot R (selecting of the target spot Tₖ) can be performed by applying a rotational or translational movement to the irradiation device 9 or the mounting 17 of the irradiation sources 16 as controlled by the controller 8. In an alternative embodiment, the irradiation sources 16 can individually be tilted with respect to the mounting 17 such that the irradiation spot R can be moved by said tilting.

After the first target point T₁ has been selected and irradiated, the second target point T₂ is selected, i.e., the irradiation spot R is moved to the second target point T₂ that is different from the first target point T₁, and is then irradiated. This is repeated for each of said plurality of different target points Tₖ defined in the hypoxic region H.

When irradiating the tumor 3 as outlined above, attention should be focused on the tumor type, its radio-sensitivity, irradiation dose and fractionation schedule, tumor volume to be targeted, and stressing tumor hypoxia. There is evidence supporting the hypothesis that RIBE and RIAE are mediated by the immune-system that probably is involved, but it is not the dominant component that determines the manifestation of those phenomena, otherwise, every single metastatic patient exposed to immunotherapy (that enhances the effects of the immune system) in addition to radiotherapy would manifest it, which is mostly not the case. According to the inventor's studies, non-targeted effects were induced in most cases, but none of the treated patients used immunotherapy. Considering how many patients are treated every day with radio-immunotherapy without manifesting non-targeted effects shows that this phenomenon is still rare despite the fact that all patients are immune-competent. Therefore, by establishing suitable conditions considering the previously mentioned factors, it is possible to intentionally induce RIAE/RIBE.

### EXAMPLES

The abovementioned system and method have been tested to prove the validity of this new strategy in terms of the induction of RIAE/RIBE in the tumor. Specifically, a small group of highly selected patients was treated by targeting exclusively the hypoxic region of their bulky tumors with high-dose radiotherapy as previously described.

The evaluation of response was performed when significant regression of symptoms was recorded (average 2 - 3 weeks). The Response Evaluation Criteria in Solid Tumors (RECIST) were used for that purpose. The average volume of the targeted hypoxic region (mean: 65.6 cm³, range: 42.6-90.2 cm³) represented about only 30% of whole bulky tumor (mean: 212.9 cm³, range: 132.5-298.2 cm³; mean diameter: 7.9 cm, range: 7-10 cm). The average maximum SUV (SUVₘₐₓ) of bulky tumors was 19.3 (range: 15.2-26.7), while it was only 2.7 in the hypoxic region (range: 1-3), which represented 15% of SUVₘₐₓ among the treated tumor.

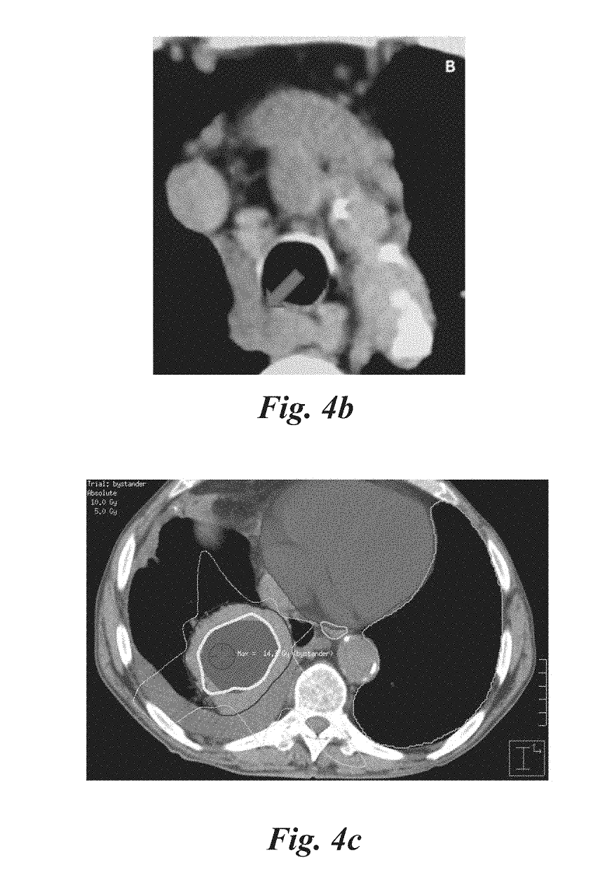

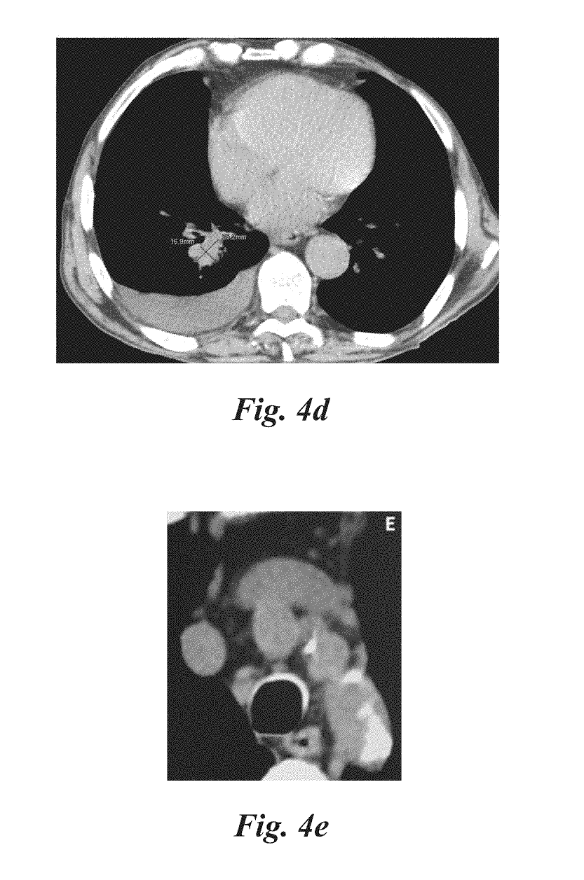

Among all patients, a significant RIBE was observed, with an intense tumor regression after an average of three weeks. Additionally, a significant RIAE was observed in about 50% of cases (FIGS. 4b and 4e). Overall, the response rate for the relief of the symptoms and mass reduction was 100%. The symptoms, for which the patients underwent radiotherapy, were under control at their last follow-up and the irradiated tumors did not re-grow. The maximum bulky downsizing (regression) was achieved after an average of four weeks with an average tumor shrinkage of 60%. No patient experienced any acute or late toxicity of any grade. No patient was treated with systemic therapy immediately prior, during, or immediately after radiotherapy so that the effects observed belong only to the radiation treatment presented herein.

The following two cases best exemplify the feasibility and efficacy of the disclosed subject-matter.

### Case 1:

A 78-year-old male with a history of prostate cancer (which had been treated with radiotherapy eight years earlier), an adenocarcinoma of the ascending colon (for which he underwent a right hemicolectomy and adjuvant chemotherapy one year earlier), now presented with a voluminous left neck metastasis, from a squamous cell carcinoma of the right ear that was previously resected in combination with a modified radical neck dissection. The 9.7 cm large tumor was un-resectable due to its infiltration of the cervical vertebra bodies. Chemotherapy was not an option. Due to increasing pain, he was treated with radiotherapy, 10 Gy in a single fraction to the 70% isodose-line (14 Gy) to the centrally located hypoxic region, corresponding to only 30% of the whole bulky tumor. Only two weeks later, about 50% of tumor regression was observed, which started from the tumor's non-irradiated periphery towards the irradiated center. The patient had no longer any pain.

### Case 2:

A 73-year-old male, previously resected for a malignant melanoma and with a history of hypothyroidism and nicotine/alcohol abuse, was diagnosed with a right lower-lung lobe squamous cell carcinoma. His tumor was very voluminous, causing dyspnea and pain. At that time, the carcinoma measured 10.9 cm (FIG. 4a). Furthermore, there were also mediastinal lymphadenopathies that, because asymptomatic, were not subjected to treatment (FIG. 4b), and two small metastases in the contralateral lung. Because of a poor performance status, both resection and systemic therapy were unsuitable. Due to the important symptoms, the multidisciplinary tumor board recommended palliative irradiation of the lung tumor. Also for this patient we chose a single 10 Gy fraction to the 70% isodose-line, prescribed to the hypoxic tumor segment (FIG. 4c). Four weeks later, a dramatic regression was observed; the tumor had reduced to 2 cm (FIG. 4d), and the dyspnea and pain completely disappeared. Furthermore, the untreated mediastinal lymphadenopathies also disappeared due the RIAE (FIG. 4e).

The disclosed subject-matter is not restricted to the specific embodiments described in detail herein, but encompasses all variants, combinations and modifications thereof that fall within the framework of the appended claims.

What is claimed is:

1. A method for treatment of a patient by irradiating a tumor by means of an irradiation device, the method comprising the following steps:
scanning at least a part of a body of the patient to generate a scan, said part comprising the tumor;
localizing the tumor in the scan;
determining a hypoxic and a normoxic region of the tumor in the scan;
targeting only the hypoxic region for irradiation; and
irradiating the targeted hypoxic region;
wherein the normoxic region is not targeted for irradiation during the treatment.

2. The method according to claim 1, the irradiation device emitting irradiation beams in different irradiation directions that intersect at the hypoxic region,
wherein said step of targeting is performed by setting an aperture of the irradiation device to match a contour of the hypoxic region of the tumor as seen in the respective irradiation direction,
wherein said step of irradiating is performed by emitting irradiation beams in the respective irradiation direction through the aperture onto the targeted hypoxic region, the aperture blocking irradiation beams from irradiating the normoxic region outside said contour, and
wherein the steps of targeting and irradiating are performed for each irradiation direction of the treatment.

3. The method according to claim 1, the irradiation device emitting irradiation beams in different irradiation directions that intersect at a single movable irradiation spot,
wherein said step of targeting is performed by moving the irradiation spot onto a target point,
wherein the step of irradiating is performed by emitting said irradiation beams in all of said different irradiation directions onto said target point, and
wherein said steps of targeting and irradiating are performed for each of a plurality of different target points defined in the hypoxic region, wherein no target points are defined in the normoxic region during the treatment.

4. The method according to claim 1, wherein a positron emission tomography is used to generate the scan.

5. The method according to claim 4, wherein a standardized uptake value of the positron emission tomography scan is calculated and the hypoxic region is determined as a region in which the standardized uptake value is equal to or smaller than 3.

6. The method according to claim 4, wherein a tracer 18F-FMISO or 18F-FDG is used for positron emission tomography.

7. The method according to claim 4, wherein a computed tomography is used to generate the scan by combining a result of the positron emission tomography with a result of the computed tomography.

8. The method according to claim 1, wherein an irradiation dose of at least 8 Gy is used for irradiation during the treatment.

9. A system for treatment of a patient by irradiating a tumor, the system comprising:
a scanner configured to scan at least a part of a body of the patient to generate a scan, said part comprising the tumor,
a processor connected to the scanner for receiving said scan, the processor being configured to localize the tumor in the scan and to determine a hypoxic and a normoxic region of the tumor in the scan,
a controller connected to the processor and to an irradiation device, the controller being configured to control said irradiation device and to target only the hypoxic region for irradiation, and
wherein the irradiation device is configured to irradiate the targeted hypoxic region, and
wherein the controller configured to not target the normoxic region for irradiation during the treatment.

10. The system according to claim 9, wherein the irradiation device is configured to emit irradiation beams in different irradiation directions that intersect at the hypoxic region,
wherein the controller is configured to target the hypoxic region by setting an aperture of the irradiation device to match a contour of the hypoxic region of the tumor as seen in the irradiation direction,
wherein the irradiation device is configured to emit irradiation beams in the irradiation direction through the aperture onto the targeted hypoxic region, the aperture blocking irradiation beams from irradiating the normoxic region outside said contour, and
wherein the controller and the irradiation device are respectively configured to perform the targeting and irradiating for each irradiation direction of the treatment.

11. The system according to claim 9, wherein the irradiation device is configured to emit irradiation beams in different irradiation directions that intersect at a single movable irradiation spot,
wherein the controller is configured target the hypoxic region by moving the irradiation spot onto a target point,
wherein the irradiation device is configured to emit said irradiation beams in all of said different irradiation directions onto said target point, and
wherein the controller and the irradiation device are respectively configured to perform the targeting and irradiating for each of a plurality of different target points defined in the hypoxic region, wherein no target points are defined in the normoxic region during the treatment.

12. The system according to claim 9, wherein the scanner is configured to use a positron emission tomography to generate the scan.

13. The system according to claim 12, wherein the processor is configured to calculate a standardized uptake value of the positron emission tomography scan and to determine the hypoxic volume as a region of the tumor in which the standardized uptake value is equal to or smaller than 3.

14. The system according to claim 12, wherein the scanner is configured to use a tracer 18F-FMISO or 18F-FDG for positron emission tomography.

15. The system according to claim 12, wherein the scanner is configured to use computed tomography to generate the scan by combining a result of the positron emission tomography with a result of the computed tomography.

16. The system according to claim 9, wherein the irradiation device is configured to use an irradiation dose of at least 8 Gy for irradiation during the treatment.
